(19) United States
(12) Patent Application Publication
(10) Pub. No.: US 2026/0098142 A1
(43) Pub. Date: Apr. 9, 2026

(54) Mechanochemical Upcycling of Polystyrene into Aromatic Compounds

(71) Applicants: Junpeng Wang, Peninsula, OH (US); Ming-Chi Wang, Akron, OH (US)

(72) Inventors: Junpeng Wang, Peninsula, OH (US); Ming-Chi Wang, Akron, OH (US)

(21) Appl. No.: 19/289,692

(22) Filed: Aug. 4, 2025

Related U.S. Application Data
(60) Provisional application No. 63/702,756, filed on Oct. 3, 2024.

(51) Int. Cl. C08J 11/16; C07C 4/22; C07C 45/46

(52) U.S. Cl. CPC C08J 11/16; C07C 4/22; C07C 45/46; C08J 2325/06

(57) ABSTRACT

A mechanochemical method for recycling polystyrene. The method includes providing polystyrene, grinding the polystyrene to thereby obtain benzene, and wherein the step of grinding the polystyrene further comprises combining AlCl₃ with the polystyrene during the grinding. The obtained benzene is then able to be recycled into styrene monomer. The obtained benzene is also able to be upcycled into benzophenone.

### CROSS REFERENCE TO RELATED APPLICATIONS

This application claims the benefit of U.S. provisional patent application Ser. No. 63/702,756, filed Oct. 3, 2024, which is incorporated by reference herein.

### STATEMENT REGARDING FEDERALLY SPONSORED RESEARCH OR DEVELOPMENT

This invention was made with government support under CHE 2204079 awarded by the National Science Foundation. The government has certain rights in the invention.

### FIELD OF THE INVENTION

One or more embodiments of the invention are directed toward a method of mechanochemically degrading polystyrene into aromatic compounds for recycling and upcycling purposes.

### BACKGROUND

Since their introduction in the 1950s, synthetic plastics derived from petroleum have achieved widespread commercial and industrial use. Among these, polystyrene (PS) has become one of the most extensively utilized plastics due to its desirable properties, including colorlessness, high mechanical strength, and chemical and thermal stability. Despite its advantages, PS remains one of the least recycled plastics, primarily due to high recycling costs and low profitability.

The global plastic industry has experienced significant growth since the mid-20th century, with polymer-based products finding application in clothing, food preservation, medical supplies, construction, automotive, and aerospace industries. In 1950, global plastic production totaled approximately 2 million metric tons; by 2021, that number had surged to approximately 390 million metric tons. This dramatic increase in production has led to a significant rise in post-consumer plastic waste. Due to the absence of a mature and economically viable recycling infrastructure, much of this waste is incinerated or relegated to landfills. Polystyrene remains a significant contributor to plastic waste. Although PS offers favorable material properties, its recycling rate remains low; in the United States, for example, the recycling rate of PS was reported to be less than 1.5% in 2015. The low rate of recycling is primarily attributed to economic and technical challenges.

Various methods, as shown in FIG. 1, have been explored for the recycling of PS. Mechanical recycling is one such method, but products derived from mechanically recycled PS often suffer from degraded performance characteristics. Alternative chemical recycling and upcycling methods have also been developed. These include post-functionalization approaches, such as alkyl-fluorination, which repurpose PS waste into value-added materials. Due to limited downstream applications, the overall impact of these strategies on reducing PS waste remains constrained. Thermochemical degradation techniques, such as pyrolysis and catalytic pyrolysis, have also been investigated. While these methods can convert PS into lower molecular weight products, they typically require high reaction temperatures (>300° C.), leading to suboptimal energy efficiency. More recently, photochemical degradation approaches have emerged, including photocatalytic oxidation methods that yield carbonyl-containing products and cascade reactions that produce diphenylmethane. Nevertheless, these methods often rely on ultraviolet light sources and organic solvents, which present challenges related to safety, cost, and scalability. Mechanochemical degradation techniques, such as ball milling, have also been reported. However, these approaches typically result in low yields of valuable monomers. For example, Balema et al. (2021) demonstrated the recovery of only 7% styrene monomer from PS via grinding in an oxygen-rich atmosphere.

There remains a need for improved methods for recycling polystyrene.

### SUMMARY

An aspect of the present invention includes a method for degrading polystyrene, the method including providing polystyrene, grinding the polystyrene to thereby obtain benzene, and wherein the step of grinding the polystyrene further comprises combining AlCl₃ with the polystyrene during the grinding.

A further aspect of the present invention includes a method for recycling polystyrene, the method including providing polystyrene, grinding the polystyrene to thereby obtain benzene, wherein the step of grinding the polystyrene further comprises combining AlCl₃ with the polystyrene during the grinding, and synthesizing styrene monomer from the obtained benzene by brominating the obtained benzene to thereby produce bromobenzene and performing an iron-catalyzed cross-coupling of the bromobenzene with vinyl acetate to thereby produce styrene monomer.

Another aspect of the present invention includes a method for upcycling polystyrene, the method including providing polystyrene, grinding the polystyrene to thereby obtain benzene, wherein the step of grinding the polystyrene further comprises combining AlCl₃ with the polystyrene during the grinding, and synthesizing styrene monomer from the obtained benzene by reacting ethane and the obtained benzene to thereby produce ethylbenzene and performing catalytic dehydrogenation of the ethylbenzene to thereby produce styrene monomer.

Yet another aspect of the present invention includes a method for upcycling polystyrene, the method including providing polystyrene, grinding the polystyrene to thereby obtain benzene, wherein the step of grinding the polystyrene further comprises combining AlCl₃ with the polystyrene during the grinding, and further combining benzoic anhydride with the AlCl₃ and the polystyrene during the grinding to thereby convert the obtained benzene into benzophenone.

### BRIEF DESCRIPTION OF THE DRAWINGS

Advantages of the present invention will become better understood with regard to the following description, appended claims, and accompanying drawings wherein:

FIG. 1 is a schematic diagram of prior art methods for recycling polystyrene;

FIG. 2 is a schematic diagram of a reaction scheme for recycling and upcycling polystyrene according to one or more embodiments of the present invention;

FIG. 3 is a plot of GPC traces of polystyrene under differing conditions;

FIG. 4 is a plot of GPC traces (THF as the eluent) of the residual polymer after various durations of grinding;

FIG. 5 is a plot of the extent of degradation against the ball milling time; and

FIG. 6 is schematic of a mechanism for mechanochemical degradation of PS according to one or more embodiments of the present invention.

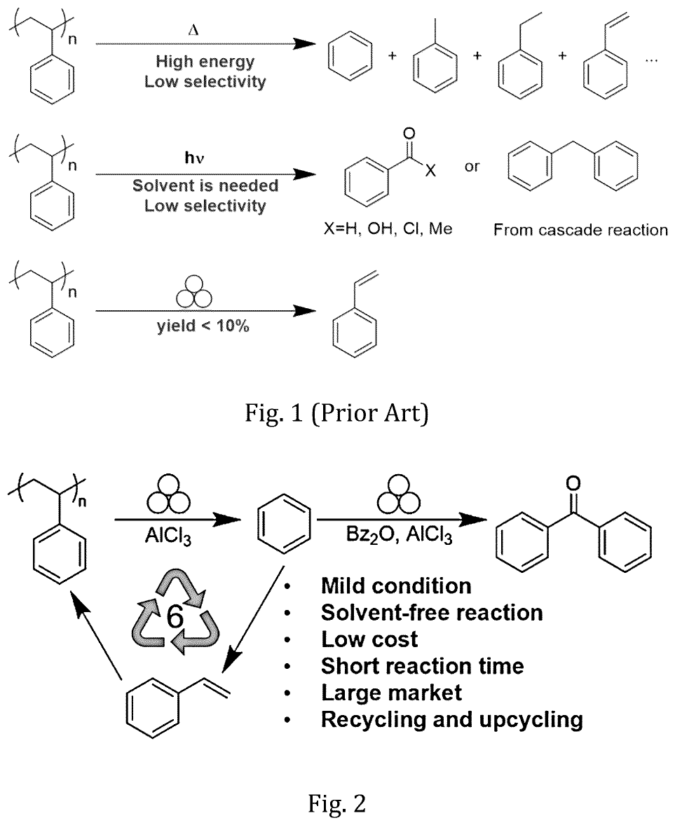

### DETAILED DESCRIPTION

### Introduction

Embodiments of the invention are based on a method of mechanochemical upcycling of polystyrene into aromatic compounds. Specifically, embodiments of the present invention provide methods of mechanochemical recycling of polystyrene (PS), and in particular embodiments the mechanochemical degradation of PS into benzene suitable for recycling and upcycling. A schematic diagram of methods according to one or more embodiments of the present invention is shown in FIG. 2. As noted above, one or more embodiments of the present invention provides a method for converting polystyrene to benzene via mechanochemical degradation. In one or more embodiments, the benzene generated in the mechanochemical degradation can be used to synthesize styrene, which can be repolymerized to produce polystyrene, allowing for the closed-loop recycling of PS. In yet other embodiments, a mechanochemical Friedel-Crafts acylation between the generated benzene and the sequentially added benzoic anhydride produces benzophenone. Embodiments of the present invention are compatible with common commercial PS products (Styrofoam boxes, utensils, and drinking cups) without removing any additives (such as foaming agents and pigments). Accordingly, embodiments of the present invention provide a mechanochemical degradation process that is solvent-free, cost-effective, and energy-efficient, and thus provides a promising route for the chemical recycling and upcycling of PS.

### Mechanochemical Degradation of Polystyrene

As shown in FIG. 2, methods of the present invention are directed towards the mechanochemical degradation of polystyrene. In one or more embodiments, mechanochemical methods for recycling polystyrene include providing polystyrene and grinding the polystyrene to thereby obtain benzene. In these and other embodiments, the step of grinding the polystyrene further comprises mixing AlCl₃ with the polystyrene. The production of benzene advantageously provides for recycling and upcycling pathways as described above, and the mechanochemical methods achieve a high conversion rate of polystyrene. In one or more embodiments the conversion rate of polystyrene is 20% or greater, in other embodiments 30% or greater, in other embodiments 40% or greater, in other embodiments 50% or greater, in other embodiments 60% or greater, in other embodiments 70% or greater, in other embodiments 80% or greater, in other embodiments 90% or greater, in other embodiments 95% or greater, and yet in other embodiments 100% or complete conversion.

Embodiments of the present invention are not particularly limited by the source of the polystyrene. For example, a number of consumer and industrial products include polystyrene such as packaging materials, disposable consumer goods, building materials, electronics and appliances, and automotive materials, amongst other goods. As embodiments of the present invention are compatible with a wide range of polystyrene goods, no pretreatment steps are required to handle additives or foaming agents that may be included with the polystyrene containing goods. Depending on the method of grinding, polystyrene goods may first be cut or shredded into smaller pieces to facilitate subsequent grinding or milling processes.

In one or more embodiments, the step of grinding the polystyrene to thereby obtain benzene, may be performed using many known grinding and milling processes and techniques.

In one or more embodiments, the step of grinding is performed without the need for a solvent. In these and other embodiments, the step of grinding performed at ambient temperature and pressure, without the need for external heating or pressurization of the mixing vessel.

In one or more embodiments, the step of grinding the polystyrene is performed using a ball mill grinder. Ball mill grinders are commonly used for the mechanical processing of solid materials through impact and attrition. The ball mill grinder typically includes a rotating cylindrical chamber partially filled with grinding media, such as stainless steel or ceramic balls, which facilitate particle size reduction as they collide with the material. Operational parameters such as milling time, rotational speed, ball-to-powder ratio, and the size of the grinding media can be adjusted to optimize energy input, collision interactions, and grinding efficiency.

In addition to ball mills, other types of mills may also be suitable for mechanochemical methods according to one or more embodiments of the present invention. These include planetary mills, vibratory mills, and attritor mills, which can provide higher energy or better scalability. Rod mills, jet mills, disc mills, and hammer mills may also be used depending on the material and processing needs. Those of ordinary skill in the art can optimize processing conditions according to the conversion percentage of polystyrene input material.

Without wishing to be bound by theory, it is believed that a longer duration of grinding promotes complete conversion of polystyrene. In one or more embodiments, the duration of the grinding step is 1 minute or greater, in other embodiments 2 minutes or greater, in other embodiments 5 minutes or greater, in other embodiments 10 minutes or greater, in other embodiments 15 minutes or greater, in other embodiments 30 minutes or greater, and in other embodiments 60 minutes or greater. In one or more embodiments, the duration of the grinding step is 60 minutes of fewer, in other embodiments, 30 minutes or fewer, in other embodiments 15 minutes or fewer, in other embodiments 10 minutes or fewer, in other embodiments 5 minutes or fewer. In one or more embodiments, the duration of the grinding step is from 1 minute to 60 minutes, in other embodiments from 2 minutes to 60 minutes, in other embodiments from 5 minutes to 60 minutes, in other embodiments from 10 minutes to 60 minutes, in other embodiments from 15 minutes to 60 minutes, in other embodiments from 30 minutes to 60 minutes.

In one or more embodiments of the present invention, the step of grinding the polystyrene further comprises mixing AlCl₃ with the polystyrene. Aluminum chloride is known to those of ordinary skill in the art as a Lewis acid catalyst in a variety of chemical reactions, including alkylation and acylation processes such as Friedel-Crafts reactions.

In one or more embodiments, the mass ratio of AlCl₃ to polystyrene (i.e. the mass of AlCl₃ to mass of polystyrene) is 0.1:1 or more, in other embodiments 0.3:1 or more, in other embodiments 0.5:1 or more, in other embodiments 0.6:1 or more, in other embodiments 0.7:1 or more, in other embodiments 0.8:1 or more, in other embodiments 0.9:1 or more, in other embodiments 1:1 or more, in other embodiments 1.1:1 or more, in other embodiments 1.2:1 or more, in other embodiments 1.3:1 or more, in other embodiments 1.4:1 or more, in other embodiments 1.5:1 or more, in other embodiments 2:1 or more, in other embodiments 2.5:1 or more, in other embodiments 3:1 or more, in other embodiments 4:1 or more, and in other embodiments 5:1. Without wishing to be bound by theory, it is believed that an excess of AlCl₃ enhances the conversion of polystyrene and decreases the amount of time required for said conversion.

Other aluminum-containing and metal chloride reagents may be used in accordance with methods of the present invention. As discussed in the Examples section, AlCl₃ advantageously provides for the highest conversion percentage of polystyrene.

### Recycling of Polystyrene and Synthesis of Styrene Monomer

According to one or more embodiments of the present invention, methods of mechanochemically recycling polystyrene include synthesizing styrene monomer from the obtained benzene. In these and other embodiments, the step of synthesizing styrene monomer from the obtained benzene includes brominating the obtained benzene to thereby produce bromobenzene and performing an iron-catalyzed cross-coupling of the bromobenzene with vinyl acetate to thereby produce styrene monomer.

The person of ordinary skill in the art appreciates that said steps may be performed according to the following scheme.

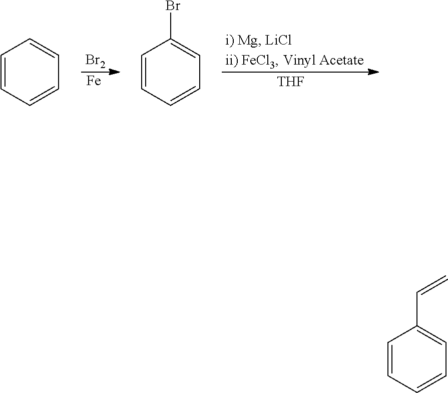

Alternative schemes may be used to convert benzene to styrene monomer. For example, in an established industrial process developed by the Dow Chemical Company and Snamprogetti Ltd., ethylbenzene is synthesized by reacting ethane with benzene, with styrene subsequently produced through catalytic dehydrogenation.

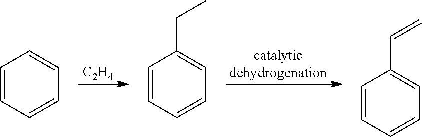

The efficient degradation of PS into benzene, and the convenient synthesis of styrene from benzene, and the established polymerization methods of styrene to form PS together, advantageously, allow for the closed-loop chemical recycling of PS to be established.

### Upcycling Polystyrene to Synthesize Benzophenone

In one or more embodiments, methods of the present invention are further directed towards the synthesis of benzophenone from polystyrene. In these and other embodiments the step of grinding the polystyrene further comprises combining benzoic anhydride with the AlCl₃ and the polystyrene during the grinding to thereby convert the obtained benzene into benzophenone. Advantageously, all the necessary compounds may be combined, mixed, and ground within the same grinding vessel which facilitates simple and facile conversion of polystyrene to benzophenone for upcycling use.

In one or more embodiments, the step of wherein the step of grinding the polystyrene comprises a two-step grinding process comprising combining the AlCl₃ and the polystyrene with a supramolecular trap and performing a first grinding to obtain benzene, and combining benzoic anhydride with the obtained benzene and performing a second grinding to obtain benzophenone. In these and other embodiments, each of the first grinding and second grinding are performed in the same mixing vessel.

Those of ordinary skill in the art readily appreciate that a supramolecular trap is a molecule or assembly of molecules designed to capture and encapsulate other molecules, effectively sequestering them. Here, trapping the benzene is advantageous for increasing the conversion efficiency to benzophenone. Suitable supramolecular traps include hexafluorobenzene (C₆F₆). Other supramolecular traps include, without limitation, octafluoronaphthalene (C₁₀F₈).

While certain advantages of embodiments of the present invention are disclosed above, other specific advantages are disclosed here. Embodiments of the present invention provide an efficient and facile degradation of polystyrene into benzene, which may then be used in recycling and upcycling applications. In particular embodiments of the present invention, methods for upcycling polystyrene into benzophenone in a one pot process.

In light of the foregoing, it should be appreciated that the present invention advances the art by providing improvements for a method of mechanochemical upcycling of polystyrene into aromatic compounds. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

### Materials and Instrumentation

The following chemicals and reagents were used in performing the following examples. The chemicals and reagents were used without further purification unless specifically noted. Polystyrene pellets (atactic, Mn˜165 kDA), benzoic anhydride (>99%), sodium bicarbonate (>99.7%), butylated hydroxytoluene (BHT), styrene, 1,3,5-trimethoxybenzene, and benzene were purchased from Sigma-Aldrich. Aluminum chloride (>99%) benzoyl peroxide (75%), and iron chloride were purchased from Fisher Scientific. 2,2,6,6-tetramethylpiperidoxyl (TEMPO, 97%) was purchased from Aaron Chemicals. Hexafluorobenzene was purchased from Synquest Laboratories. Octafluoronaphthalene was purchased from Ambeed. CDCl₃ was purchased from Cambridge Isotope Laboratories, Inc. syrene-d₃ was purchased from Santa Cruz Biotechnology, Inc. Styrene and syrene-d₃ were passed through a short plug of basic alumina to remove inhibitor prior to use for polymerization. Other chemicals were used as received.

All ball mill grinding experiments were performed with a Retsch ball mill including a stainless steel jar (50 mL) and multiple stainless steel balls (diameters=12.7 mm). The grinding experiments were conducted at ambient temperature using a frequency of 30 Hz.

Gel permeation chromatography (GPC) experiments were carried out using a Tosoh EcoSEC HLC-8320 GPC, which comprises two 17393 TSKgel columns (7.8 mm ID×30 cm, 13 μm) and one 17367-TSKgel Guard Column (7.5 mm ID×7.5 cm, 13 μm). The chromatography was conducted using preservative-free HPLC grade THF (obtained from Fisher Chemical) at a flow rate of 1 mL min⁻¹ at 40° C. A high temperature gel permeation chromatography (HT-GPC) was also used for analysis of the aliphatic component in the degradation products. Analyses were performed using a Tosoh EcoSEC HLC-8321 GPC RI Detector. Columns consisted of one TSKgel Hhr (30) HT2 Guard Column (7.5 mm ID×7.5 cm, 30 μm), two TSKgel GMHhr-H (20) HT2 columns (7.8 mm ID×30 cm, 20 μm), and one TSKgel Hhr HT-RC reference column (7.8 mm ID×30 cm, 13 μm). HPLC grade 1,2,4-trichlorobenzene (0.1 wt. % BHT added as stabilizer) was used as the eluent at a flow rate of 1 mL min⁻¹ at 140° C. Data was measured relative to polystyrene standards.

MALDI-MS spectra were recorded on a Bruker UltrafleXtreme MALDI-ToF/ToF mass spectrometer (Bruker, Billerica, MA) equipped with a Nd:YAG laser emitting at 355 nm in positive reflectron mode. Each sample was calibrated using 2 k and 4 k PMMA. FlexAnalysis v3.4 was used as processing software. The sample was dissolved in methanol at a concentration of 10 mg/mL and was analyzed using DCTB as matrix and sodium trifluoroacetate (NaTFA) as cationizing salt. Matrix and salt were dissolved in the same solvent at 20 mg/mL and 10 mg/mL, respectively. The sample, matrix, and salt solution were mixed in the ratio 10:2:1 (v/v/v), and approximately 0.5 μL of the final mixture was spotted on the sample plate for MALDI-MS analysis.

### Degradation Study—Grinding PS and AlCl₃

Initially, 208 mg PS and 208 mg AlCl₃ along with six stainless steel balls with diameters of 12.7 mm were added to a 50-mL stainless steel jar of a ball mill, and the milling was conducted at 30 Hz. After 60 minutes of ball milling, CDCl₃ was added directly to the stainless steel jar. The resulting supernatant was collected and analyzed by both ¹H NMR and ¹³C NMR.

Benzene was confirmed as the only aromatic product formed during mechanochemical degradation. Specifically, ¹H NMR and ¹³C NMR spectra of virgin PS and the degradation product after 60 min of ball milling were compared. The spectra were obtained from a minimal CDCl₃ extraction (˜700 μL), which was sufficient only for NMR analysis. Notably, no peaks corresponding to PS were observed in the ¹H NMR or ¹³C NMR, indicating complete degradation. Meanwhile, the ¹H NMR peak at 7.36 ppm and the ¹³C NMR peak at 128.49 ppm are consistent with the formation of benzene. The results demonstrated that benzene is the only aromatic product formed during the mechanochemical degradation.

In addition, peaks at the aliphatic region (1.3-0.7 ppm) can be assigned to the aliphatic products after the phenyl groups are detached. The starting polymer and degradation products were characterized with GPC at 140° C. with 1,2,4-trichlorobenzene as the eluent, in which the refractive index is positive for aliphatic products but is negative for PS. The change of refractive index from negative to positive is consistent with the conversion of PS into aliphatic products. Other degradation products were identified using the product was subject to matrix-assisted laser desorption/ionization-mass spectrometry (MALDI spectrum analysis). Ball milling of 165 kDA PS pellets was produced degradation products. MALDI analysis showed major peaks in the m/z range of 170-250. These peaks were assigned to C13, C14, and C17 aliphatic products. Thus, it was confirmed that aliphatic products were produced in addition to the aromatic benzene.

Due to the volatility of benzene, it is challenging to quantify the extent of degradation based on the yield of benzene. Therefore, the extent of degradation was quantified based on the consumption of PS as determined by ¹H NMR integration using 1,3,5-trimethoxybenzene as standard. Thus, the extent of degradation after ball milling was determined by adding DCM (30 mL×3) to the grinding mixture, and the mixture was filtered via vacuum filtration. To the filtrate was added 5 mol % (relative to the number of moles of PS) 1,3,5-trimethoxybenzene or butylated hydroxytoluene as an internal standard for ¹H NMR. The solution was dried and subjected to NMR and GPC characterization. The PS was ground in a ball mill for 1 min. The proton peaks of 1,3,5-trimethoxybenzene are were highlighted, and the ortho-proton peaks were identified and the integrations of the proton peaks of 1,3-5-trimethoxybenzene were set to be 1. The integrations of the ortho-protons peaks before and after mechanochemical degradation were x and y, respectively. Conversions were then calculated as:

$$Conversion = ( 1 - \frac{y}{x} ) \times 100 \%$$

The heterogeneity of the ground products was investigated by dissolving PS (208 mg, 2 mmol) and 1,3,5-trimethoxybenzene (16.8 mg, 0.1 mmol) in DCM, drying the solution, and adding to the ball mill jar. AlCl₃ (208 mg) was then added and the mixture was ground for 5 minutes. The grinding products from 5 different locations of the ball mill, including 2 portions from the lid (L1, L2), 1 portion from stainless steel balls (B1), and 2 portions from the jar (J1, J2), were collected. The partial spectrum of PS before ball milling was also studied. Different locations showed different NMR integrations, indicating the inhomogeneity of the ground sample.

The number of balls and the amount of AlCl₃ used in the ball milling were varied to optimize the efficiency of degradation. Table 1 summarizes the results of these studies.

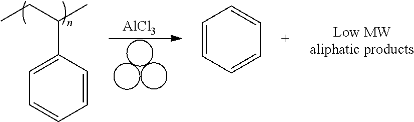

TABLE 1
Optimization of the grinding parameters for
the mechanochemical degradation of PS.

| | | | | | Extent of |
|---|---|---|---|---|---|
| | PS | AlCl₃ | Time | Number | degradation |
| Entry | (mg) | (mg) | (min) | of balls | (%)^{[b]} |
| 1 | 208 | 208 | 60 | 2 | 71 |
| 2 | 208 | 208 | 60 | 4 | 73 |
| 3 | 208 | 208 | 60 | 6 | 100 |
| 4 | 208 | 208 | 15 | 6 | 89 |
| 5 | 208 | 104 | 15 | 6 | 38 |
| 6 | 208 | 52 | 15 | 6 | 14 |
| 7 | 208 | 52 | 120 | 6 | 100 |

When the milling was conducted with six balls and 1:1 AlCl₃ to PS (in weight ratio) for 60 min, a full conversion was achieved. When two and four balls were used while keeping other conditions identical, the extents of degradation were 71% and 73%, respectively. These findings imply that a greater number of balls in the jar facilitates more collision events during milling. The marginal increase in the extent of degradation from two balls to four balls could be attributed to the altered trajectory of the moving balls when using four balls, which reduces the number of collisions and offsets the increase in the number of balls. Grinding with more than six balls was not attempted as per the recommendations in the manual for the ball mill. Moreover, when the ball milling time was reduced showed different NMR integrations, indicating the inhomogeneity of the ground sample.

### Degradation Study—Grinding PS and Other Additives

In accordance with the foregoing, further experiments were performed using a number of reagents included in Table 2. Reagents generally were selected from aluminum compounds and metal chlorides. All experiments were done with 208 mg PS, 208 mg reagents, 6 stainless steel grinding balls, and grinding for 15 min at grinding frequency of 30 Hz.

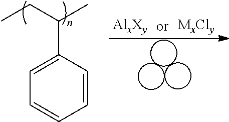

TABLE 2
Mechanochemical degradation of PS with various reagents

| Entry | Reagent | Conv. (%) |
|---|---|---|
| 0 | N/A | 10 |
| 1 | AlCl₃ | 88 |
| 2 | InCl₃ | 39 |
| 3 | SnCl₂ | 17 |
| 4 | TiCl₂ | 30 |
| 5 | ZnCl₂ | 19 |
| 6 | FeCl₃ | 29 |
| 7 | Al₂O₃ (basic) | 29 |
| 8 | Al₂O₃ (neutral) | 31 |
| 9 | Al₂O₃ (acidic) | 32 |

To further understand the mechanochemical degradation process, a kinetic study was performed in which the ball milling was conducted for various periods of time, and the resulting mixtures were characterized using ¹H NMR. Grinding experiments were performed in triplicate for each duration. The corresponding extents of degradation are summarized in Table 3. All experiments were conducted using 208 mg PS and 208 mg AlCl₃. Number of balls=6. Grinding frequency=30 Hz. The extent of degradation corresponds with the conversion percentage as calculated above. Specifically, ¹H NMR spectra and the peaks 6.10 ppm and 3.78 ppm correspond to those of 1,3,5-trimethoxybenzene, which was used as a standard to determine the extent of degradation.

TABLE 3
Extents of degradation for the kinetic study
of mechanochemical degradation of PS

| Grinding time | Extent of degradation |
|---|---|
| (min) | (%) |
| 1 | 30 |
| 1 | 31 |
| 1 | 35 |
| 3 | 53 |
| 3 | 53 |
| 3 | 48 |
| 5 | 59 |
| 5 | 74 |
| 5 | 61 |
| 10 | 86 |
| 10 | 81 |
| 10 | 85 |
| 15 | 89 |
| 15 | 89 |
| 15 | 88 |
| 30 | 96 |
| 30 | 97 |
| 30 | 94 |
| 60 | 100 |
| 60 | 100 |
| 60 | 100 |

Remarkably, the extents of degradation reached 32%, 51%, and 65% at 1 min, 3 min, and 5 min of ball milling, respectively. The degradation rate is notably faster than that of any previously reported PS degradation methods, and, therefore, provides an advantage in terms of energy efficiency.

The molecular weight of each sample was characterized with GPC. When trichlorobenzene is used as the eluent, the partially degraded samples would contain both negative RI (PS) and positive RI (aliphatic) components. This can lead to signal cancellation between the two, resulting in inaccurate reading. To assist the comparison across different ball milling periods, THF was used as the eluent here to ensure that the refractive indices are positive for all samples. In the initial 5 min of milling, little change was observed in the molecular weight of the residual polymer despite the substantial extent of degradation detected by ¹H NMR. The observation of little change in the MW of the residual polymer within the first 5 min of ball milling suggests that an activation step is required before the AlCl₃ catalyst can effectively react with PS. Once this activation barrier is overcome, the reaction proceeds rapidly, resulting in both benzene elimination and a significant reduction in MW. This behavior leads to a mixture: some PS chains remain unreacted, while others undergo rapid degradation to yield benzene and low MW aliphatic products.

### Kinetic Study—Milling Variance

To further understand the mechanochemical degradation process, a kinetic study was performed in which the ball milling was conducted for various periods of time, and the resulting mixtures were characterized using ¹H NMR. To gain further insight into the degradation process, a mass balance study was performed. Polystyrene (208 mg, 2 mmol) and AlCl₃ (208 mg, 1.56 mmol) were added into a ball milling jar and ground for various durations. After ball milling, diethyl ether (20 mL×5) was added to the grinding mixture, which was then filtered through a plug of basic aluminum oxide. The filtrate was dried under vacuum until no further weight change was observed, and the residual mass was measured. The result is presented in Table 4.

$$Theoretical mass = 208 \times \frac{( 100 - x )}{x} + 52 \times \frac{x}{100} , where x is \% conversion$$

TABLE 4
Mass residual after different periods of grinding time.

| Time^{a} | Conversion | Theoretical | Experimental |
|---|---|---|---|
| (min) | (%) | mass (mg) | mass residual (mg) |
| 40 | 97 | 56 | 55 ± 4.8 |

This process recovered 55 mg of soluble polymer backbone (lacking aromatic signals by NMR), corresponding to 98% of the mass of the PS backbone. Through a mechanochemical cascade Friedel-Crafts acylation with benzoic anhydride, obtained 142 mg of benzophenone, accounting for 39% of the aromatic rings in the original PS. The remaining 61% was likely lost as volatile products (e.g., benzene) or formed insoluble residues.

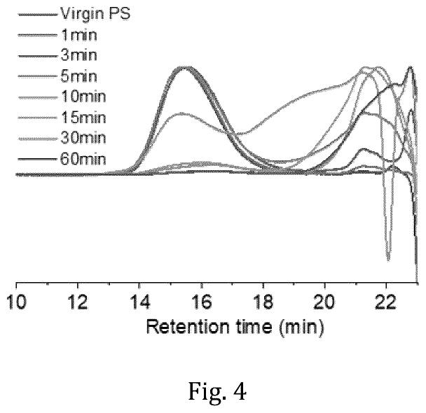

As shown in FIG. 4, the GPC traces (THF as the eluent) of the residual polymer after various durations of grinding when using 208 mg PS/208 mg AlCl₃, six balls, and a grinding frequency of 30 Hz. The GPC chromatograms in the inset were normalized to 1 at the main peak for ease of comparison.

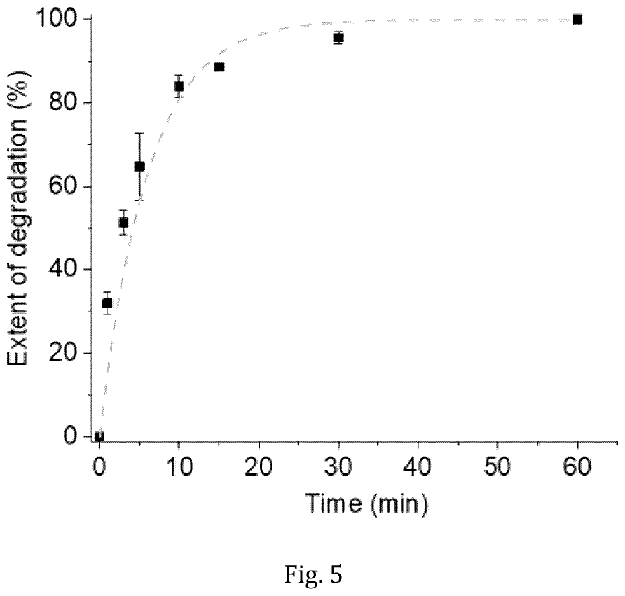

As shown in FIG. 5, the extent of degradation (calculated based on ¹H NMR) is plotted against the ball milling time. The GPC chromatograms were normalized to 1 at the main peak for ease of comparison. The extent of degradation was quantified based on the consumption of PS as determined by ¹H NMR integration using 1,3,5-trimethoxybenzene as standard.

### Determining the Effect of Molecular Weight

In addition to the 165 kDa PS pellets, a 20 kDa PS was prepared through nitroxide-mediated polymerization. A 50 mL Schlenk flask equipped with a stir bar was charged with the initiator, benzoyl peroxide (145.2 mg, 0.6 mmol, 1 equiv.), styrene (34.2 mL, 300 mmol, 500 equiv.), and TEMPO (140 mg, 0.9 mmol, 1.5 equiv.). The flask was sealed, and the solution was deoxygenated via three freeze-pump-thaw cycles, and then backfilled with nitrogen. The flask was placed in a pre-heated oil bath (110° C.) and the reaction mixture was stirred for 24 h until 40% degradation was reached. The flask was then opened to air and the solution was diluted with DCM. The polymer was precipitated in cold methanol (2×) and then thoroughly dried under vacuum to afford a white powder (10 g, 32% yield). Mₙ=20 kDa, Ð=1.18.

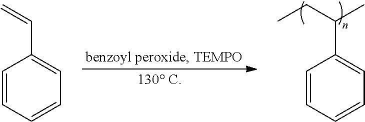

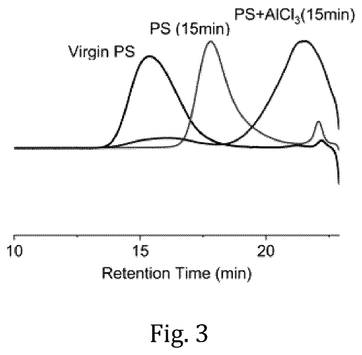

The 20 kDa PS was subjected to ball milling (six balls, 100 wt % AlCl₃). The 20 kDa PS showed an even faster degradation rate, with 80% degradation after 2 min of ball milling and nearly complete conversion after 4 min of ball milling. As shown in FIG. 3, GPC traces for virgin PS, PS ground without AlCl₃ and PS ground with AlCl₃ are provided.

### Mechanistic Study of Mechanochemical Degradation of PS

It was previously proposed that in a thermal degradation of PS in the presence of AlCl₃ that a cationic degradation mechanism controlled the degradation. Specifically, AlCl₃ reacts with moisture to generate a Brønsted acid, which protonates the phenyl ring, leading to the formation of a Wheland intermediate. Isotope labelling experiments were performed using the synthesis of PS-d₃.

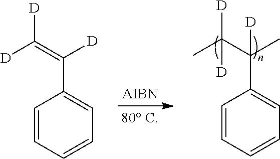

Styrene-d₃ (288 uL, 2.5 mmol, 100 equiv). was dissolved in 1 mL DCM, and the solution was passed through a basic alumina plug to remove inhibitor. The monomer solution was transferred to a vial charged with a stir bar, and DCM was removed by rotatory evaporator. To the vial was added AIBN (4.1 mg, 0.025 mmol, 1 equiv). The solution was bubbled with N₂ for 5 min. The vial was then placed in a pre-heated oil bath (80° C.), and the reaction mixture was stirred for 16 h. The flask was opened to air, and the solution was diluted with DCM. The polymer was precipitated in cold methanol 2 times and then thoroughly dried under vacuum to afford a white powder (200 mg, 77% yield). Mₙ=20 kDa, Ð=1.9. When 10 μL D₂O was added to the milling mixture, both C₆H₅D and C₆H₆ were observed, with relative fractions of 46% and 54%, respectively, confirming that ambient water can provide the proton to form benzene. When PS-d₃ was ball milled with AlCl₃ in the absence of D₂O, both C₆H₅D and C₆H₆ were observed, and their fractions were 30% and 70%, respectively, based on the integrations of the corresponding peaks in 13C NMR. In this case, both ambient water and the polymer backbone provided proton for benzene.

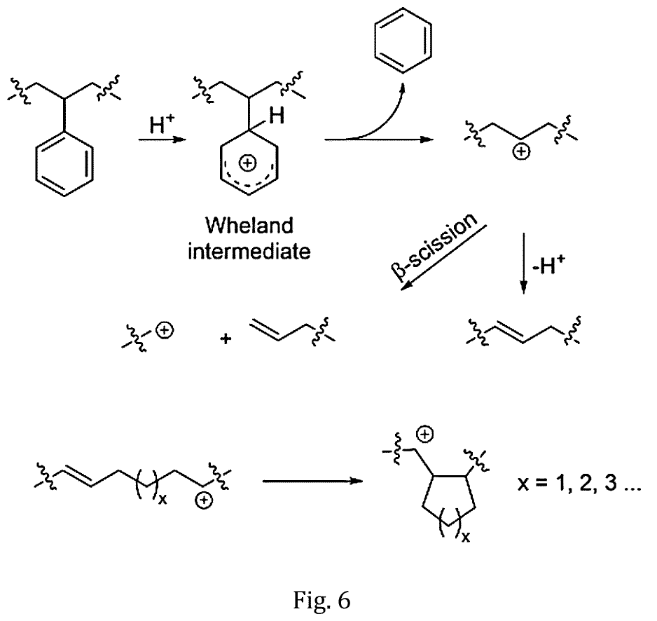

The generation of proton from the polymer backbone indicates the occurrence of deprotonation during the degradation process, as shown in FIG. 6, which would lead to the formation of alkene. In addition, the molecular weight of PS decreased much faster when it was milled with AlCl₃ than when only PS is milled; the substantial reduction in molecular weight is likely an outcome of β-scission, which would also cause the generation of alkene. Notably, though, alkene was not observed in this study. It was hypothesized that the alkenes generated from deprotonation and β-scission of the carbocation could be consumed by the carbocations through cyclization. An extension of this hypothesis is that preexisting alkenes can also be consumed during the mechanochemical degradation process. To test this idea, a mechanochemical degradation on a styrene-butadiene-styrene (SBS) triblock copolymer was performed. Significantly, it was observed that the olefin peak in SBS diminished and ultimately disappeared during the degradation process. This result confirmed that alkenes could be consumed, presumably by the generated carbocations to form cyclic structures. The formation of cyclic structures is also supported by MALDI-MS results, which showed products with degree of unsaturation of 2-3.

Synthesis of Styrene from Benzene

A notable benefit of obtaining benzene through mechanochemical degradation is its utility in the production of styrene monomer. For example, in an established industrial process developed by the Dow Chemical Company and Snamprogetti Ltd., ethylbenzene is synthesized by reacting ethane with benzene, with styrene subsequently produced through catalytic dehydrogenation. A laboratory two-step synthesis is also demonstrated here: bromination of benzene afforded bromobenzene, which then underwent an iron-catalyzed cross-coupling with vinyl acetate to generate styrene according to the following scheme.

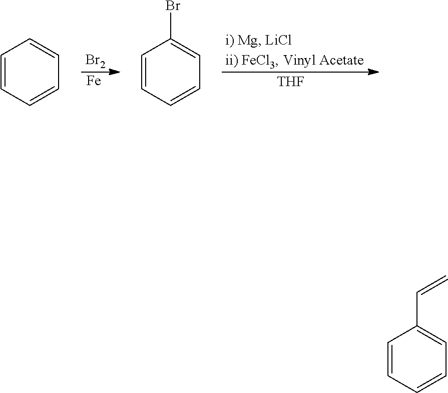

The efficient degradation of PS into benzene, the convenient synthesis of styrene from benzene, and the established polymerization methods of styrene to form PS together allow for the closed-loop chemical recycling of PS to be established.

Bromination was performed by adding benzene (4.68 g, 60 mmol, 1 equiv.) and iron powder (0.168 g, 3 mmol, 0.05 equiv.) to a round bottom flask. The flask was placed in an ice/water bath (0° C.), and to the flask, bromine (3.8 mL, 12 g, 75 mmol, 1.25 eq) was added dropwise with an additional funnel. After the addition of bromine, the reaction mixture was allowed to warm to room temperature and was then stirred for 2 days. Reaction is quenched with NaOH aqueous solution followed by washing with brine three times. A yield of 63% was determined by ¹H NMR with BHT as a standard.

Iron-Catalyzed cross-coupling was then performed. A Schlenk tube was charged with Mg chips (0.3 g, 12.5 mmol, 1.25 equiv.) and anhydrous LiCl (0.53 g, 12.5 mmol, 1.25 equiv.) and was heated under vacuum for 10 min. The Schlenk tube was purged with nitrogen and placed in an ice/water bath (0° C.), and to the tube, bromobenzene (1.57 g, 10 mmol, 1 equiv.) in anhydrous THF was added using a syringe. The mixture was stirred for 2 h before it was allowed to warm to room temperature. After another 2 h of stirring, the mixture was cooled again in an ice/water bath, and a solution of FeCl₃ (81 mg, 0.5 mmol, 0.05 equiv.) in anhydrous THF was added via a syringe, followed by the addition of vinyl acetate (861 mg, 10 mmol, 1 equiv.). The mixture was stirred at 0° C. for 3 h before being quenched with saturated aqueous NH₄Cl solution. The mixture was extracted with ethyl acetate, and the combined organic layers were dried over MgSO₄, and the solvent was removed under reduced pressure. The residue was subjected to ¹H NMR (with BHT as standard) and a yield of 98% was determined.

### Mechanochemical Friedel-Craft Acylation of Benzene

The generation of benzene from mechanochemical degradation of PS also provides a route of upcycling by coupling with other mechanochemical reactions. Based on the understanding of previous mechanochemical Friedel-Crafts acylation work, it was hypothesized that benzene generated from the mechanochemical degradation of PS with AlCl₃ could undergo Friedel-Crafts acylation with benzoic anhydride, forming benzophenone. Previous mechanochemical Friedel-Crafts acylation reported by others was performed on other aromatic substrates but not on benzene. Thus, to confirm the feasibility of generating benzophenone through mechanochemical Friedel-Crafts acylation of benzene, the reaction was tested using benzene as the substrate.

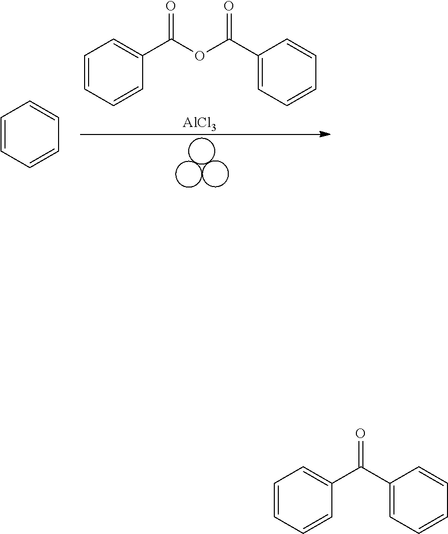

Benzene, benzoic anhydride and AlCl₃ were placed in the stainless-steel jar, grinding 30 min for each cycle. Cycles were repeated until the reaction was done. After the reaction, all reaction mixture was transferred into a beaker with excess amount of water to quench the reaction and liberate the product, benzophenone. After stirring for half 1 h, the product was extracted out with dichloromethane. Solvent was removed by rotatory evaporator. The mixture was further purified by column chromatography with 5% ethyl acetate in hexane. The results of these experiments are included in Table 5. For each grinding experiment, 600 μL benzene (6.7 mmol) and 1524.4 mg of benzoic anhydride (6.7 mmol) were used.

TABLE 5
Yields of benzophenone for various grinding parameters
used in the mechanochemical Friedel-Craft acylation

| | | Grinding | Number of | |
|---|---|---|---|---|
| Entry | AlCl₃ (mg) | time (min) | balls | Yield (%) |
| 1 | 2246.3 | 120 | 2 | 61 |
| 2 | 2246.3 | 120 | 4 | 70 |
| 3 | 2246.3 | 120 | 6 | 79 |
| 4 | 1123.1 | 120 | 6 | 4 |
| 5 | 561.6 | 120 | 6 | 0.4 |
| 6 | 2246.3 | 60 | 6 | 64 |
| 7 | 2246.3 | 240 | 6 | 72 |

As shown in Table 5, the number of milling balls varied. As the number of balls was increased from two to six, the isolated yield of benzophenone also increased from 61% to 79%, likely due to an increase in collision events. The amount of AlCl₃ exhibited an even more pronounced effect on the mechanochemical Friedel-Crafts acylation. When less than 1 equiv. AlCl₃ was used, the yield of benzophenone remained below 5%, whereas increasing the amount of AlCl₃ to 2.25 equiv. resulted in a yield of benzophenone up to 80%. A grinding cycle of 30 min was used, and experiments were conducted for two, four, and eight grinding cycles. The isolated yields consistently ranged between 60% and 80%, suggesting that two cycles of grinding were sufficient for the reaction.

### Mechanochemical Cascade Reaction

With these results in hand, envisioning a one-pot reaction that degrades PS into benzene and then converts benzene into benzophenone.

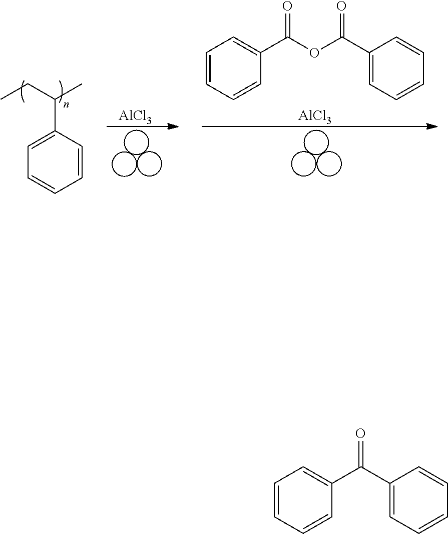

Accordingly, benzoic anhydride was added together with PS and an excess amount of AlCl₃ and ground in a ball mill for 3 h. The yield of benzophenone was only 11%, and around 20% of PS remained, suggesting that benzoic anhydride could affect the AlCl₃-assisted mechanochemical degradation of PS.

To address the compatibility problem, it was investigated whether other conditions would affect the conversion. Specifically, a mixture of PS (208 mg, 2 mmol) and AlCl₃ (208 mg, 1.56 mmol) was added to the ball milling jar and was ground for 15 min. Benzoic anhydride (452 mg, 2 mmol) and AlCl₃ (600 mg, 4.5 mmol) were then added to the ball milling jar, and the mixture was ground for a period (30 min, 60 min, 90 min, or 120 min). After the ball milling, the reaction mixture was transferred into a flask with excess amount of water. After stirring for 0.5 h, the product was extracted with dichloromethane. Further purification was achieved by column chromatography with 5% ethyl acetate in hexane. The isolated yield was found to be close to the yield determined by comparing the integration of benzophenone with that of BHT in the crude ¹H NMR, and thus all the further reported yields for the mechanochemical cascade reaction were obtained by using BHT as the standard. After using the optimized condition (100 wt % AlCl₃ and six balls) for 15 min and then added benzoic anhydride and additional AlCl₃ into the ball milling jar and continued ball milling for another 60 min. This also gave a yield of 11%, but with no PS remaining, as shown in Table 6.

TABLE 6
Yields of benzophenone for various conditions of mechanochemical
degradation followed by Friedel-Crafts acylation

| Entry | Condition | Yield (%) |
|---|---|---|
| 1 | two-step addition | 11 |
| 2 | liquid nitrogen applied before | 15 |
| | the second step | |
| 3 | placing the milling jar in | 9 |
| | freezer before the second step | |
| 4 | the second step in DCM | 0 |
| | (thermal) | |
| 5 | the second step in DCM with | 0 |
| | Bz₂O (thermal) | |
| 6 | adding C₆F₆ in the first step | 37 |
| 7 | adding C₆F₆ in the second | 19 |
| | step | |
| 8 | adding C₆F₆ in the first step | 39 |
| | and using Teflon tape to wrap | |
| | the joint | |
| 9 | increasing the scale by three | 23 |
| | times without adding C₆F₆ | |
| 10 | increasing the scale by three | 12 |
| | times with adding C₆F₆ added | |
| 11 | 1 min on/off cycles without | 11 |
| | C₆F₆ | |
| 12 | 1 min on/off cycles with C₆F₆ | 12 |
| 13 | adding C₁₀F₈ in the first step | 20 |

Considering the near-complete degradation achieved in the initial step and the high isolated yield obtained in the second step model reaction, two possible factors were identified: (1) the evaporation of benzene during grinding and (2) the effect of the viscosity of the polymer/oligomer on the efficiency of the mechanochemical Friedel-Crafts acylation. To diagnose the problem, following the mechanochemical degradation of PS in the ball mill, the product was transferred along with benzoic anhydride and additional AlCl₃ to a flask. DCM was added to the mixture, and the reaction was allowed to proceed thermally for 16 h. However, neither benzophenone nor diphenyl methane (resulting from the Friedel-Crafts alkylation of benzene with DCM) was observed, suggesting that benzene had evaporated before the thermal reaction. The one-pot mechanochemical process was then studied and an attempt to prevent the evaporation of benzene by cooling the ball milling jar using liquid nitrogen and storing it in a freezer before adding benzoic anhydride and additional AlCl₃ to initiate the Friedel-Crafts acylation was pursued. The overall yield of benzophenone remained largely unaffected. This indicates that most of the benzene had already evaporated during the mechanochemical degradation.

Accordingly, it was proposed that addition of a supramolecular trap such as hexafluorobenzene (C₆F₆) could reduce the loss of benzene. Adding C₆F₆ in the first step led to a 39% yield of benzophenone, see Table 6, above. Moreover, the addition of C₆F₆ in the second step only produced a 19% yield of benzophenone, further confirming that benzene evaporation during the first step was responsible for the low yield. Other strategies used in an attempt to retain benzene included wrapping the joint of the ball milling jar with Teflon tape or parafilm, using supramolecular trap octafluoronaphthalene (C₁₀F₈) instead of C₆F₆, enlarging the scale, and dividing the grinding time in the first step into multiple on/off cycles to reduce heat accumulation, but none of these attempts further boosted the yield.

Aside from PS pellets, the same reaction condition—i.e., one-pot degradation with two sequences of addition—was applied to commercial PS products including Styrofoam boxes, spoons, and drinking cups. The commercial PS products were cut into small pieces to fit in the milling jar, and no other pretreatment was done to the PS products. The reactions for all products provided yields that were comparable to the yield obtained from that for PS pellets. Specifically, PS pellets had a 39% yield, PS spoons had a 38% yield, Styrofoam had a 40% yield, and PS drinking cups had a 48% yield. These results suggest that degradation of PS and the mechanochemical Friedel-Crafts acylation are compatible with the additives used in these commercial PS products, such as pigments and foaming agents. Because of the compatibility, purification steps to remove the additives are not needed. These results further highlights the practicality of using the method demonstrated here.

In total, the experiments contained herein demonstrate the mechanochemical degradation of PS into benzene via ball mill grinding. The entire reaction is solvent-free, which is beneficial for it to be scaled up to industrial process. The mild conditions, short reaction time, and inexpensive reagent further highlight the economic value of this process. The generated benzene can be used to resynthesize styrene monomer, which can be employed in the production of PS, making it possible to achieve a closed-loop chemical recycling of PS. The benzene produced during the mechanochemical degradation step can also be directly used for mechanochemical Friedel-Crafts reaction to produce benzophenone. Lastly, the hydrocarbon backbone can potentially be recovered as aliphatic products for applications such as lubricants and releasing agents.

Various modifications and alterations that do not depart from the scope and spirit of this invention will become apparent to those skilled in the art. This invention is not to be duly limited to the illustrative embodiments set forth herein.

What is claimed is:

1. A mechanochemical method for degrading polystyrene, the method comprising:
providing polystyrene;
grinding the polystyrene to thereby obtain benzene; and
wherein the step of grinding the polystyrene further comprises combining AlCl₃ with the polystyrene during the grinding.

2. The method of claim 1, wherein the step of grinding the polystyrene is performed using a ball mill grinder.

3. The method of claim 1, wherein the step of grinding the polystyrene is performed in the absence of a solvent.

4. The method of claim 1, wherein the mass ratio of AlCl₃ to polystyrene is from 0.1:1 or greater.

5. The method of claim 4, wherein the mass ratio of polystyrene to AlCl₃ is from 1:1 or greater.

6. The method of claim 1, where the step of grinding the polystyrene takes place at ambient pressure and temperature.

7. The method of claim 1 further comprising synthesizing styrene monomer from the obtained benzene.

8. The method of claim 7, wherein synthesizing styrene monomer from the obtained benzene comprises:
brominating the obtained benzene to thereby produce bromobenzene; and
performing an iron-catalyzed cross-coupling of the bromobenzene with vinyl acetate to thereby produce styrene monomer.

9. The method of claim 7, wherein synthesizing styrene monomer from the obtained benzene comprises:
reacting ethane the obtained benzene to thereby product ethylbenzene; and
performing catalytic dehydrogenation of the ethylbenzene to thereby product styrene monomer.

10. The method of claim 1, wherein the step of grinding the polystyrene further comprises combining benzoic anhydride with the AlCl₃ and the polystyrene during the grinding to thereby convert the obtained benzene into benzophenone.

11. The method of claim 10, wherein the step of grinding the polystyrene comprises a two-step grinding process comprising:
combining the AlCl₃ and the polystyrene with a supramolecular trap and performing a first grinding to obtain benzene; and
combining benzoic anhydride with the obtained benzene and performing a second grinding to obtain benzophenone.

12. The method of claim 11, wherein the first grinding and the second grinding are performed in the same grinding vessel.

13. The method of claim 1, wherein a conversion rate of the polystyrene is 20% or greater.

14. The method of claim 1, further comprising recovering aliphatic products.

15. The method of claim 1, wherein the polystyrene comprises a polystyrene article including one or more additives.

16. The method of claim 15, wherein the polystyrene article undergoes no chemical pretreatment prior to the step of grinding.

17. The method of claim 16, wherein the polystyrene article is shredded before the step of grinding.
